SEQUENCE LISTING (106 sequences)

SEQ ID NO: 1 - DNA, 23 bp, Homo sapiens
  primer
caggtgcagctggtgcagtctgg

SEQ ID NO: 2 - protein, 7 aa, Homo sapiens
QVQLVQS

SEQ ID NO: 3 - DNA, 23 bp, Homo sapiens
  primer
caggtcaacttaagggagtctgg

SEQ ID NO: 4 - protein, 7 aa, Homo sapiens
QVNLRES

SEQ ID NO: 5 - DNA, 23 bp, Homo sapiens
  primer
gaggtgcagctggtggagtctgg

SEQ ID NO: 6 - protein, 7 aa, Homo sapiens
EVQLVES

SEQ ID NO: 7 - DNA, 23 bp, Homo sapiens
  primer
caggtgcagctgcaggagtcggg

SEQ ID NO: 8 - protein, 7 aa, Homo sapiens
QVQLQES

SEQ ID NO: 9 - DNA, 23 bp, Homo sapiens
  primer
gaggtgcagctgttgcagtctgc

SEQ ID NO: 10 - protein, 7 aa, Homo sapiens
EVQLLQS

SEQ ID NO: 11 - DNA, 23 bp, Homo sapiens
  primer
caggtacagctgcagcagtcagg

SEQ ID NO: 12 - protein, 7 aa, Homo sapiens
QVQLQQS

SEQ ID NO: 13 - DNA, 24 bp, Homo sapiens
  primer
tgaggagacggtgaccagggtgcc

SEQ ID NO: 14 - protein, 8 aa, Homo sapiens
GTLVTVSS

SEQ ID NO: 15 - DNA, 24 bp, Homo sapiens
  primer
tgaagagacggtgaccattgtccc

SEQ ID NO: 16 - protein, 8 aa, Homo sapiens
GTMVTVSS

SEQ ID NO: 17 - DNA, 24 bp, Homo sapiens
  primer
tgaggagacggtgaccagggttcc

SEQ ID NO: 18 - protein, 8 aa, Homo sapiens
GTLVTVSS

SEQ ID NO: 19 - DNA, 24 bp, Homo sapiens
  primer
tgaggagacggtgaccgtggtccc

SEQ ID NO: 20 - protein, 8 aa, Homo sapiens
GTTVTVSS

SEQ ID NO: 21 - DNA, 23 bp, Homo sapiens
  primer
gacatccagatgacccagtctcc

SEQ ID NO: 22 - protein, 7 aa, Homo sapiens
DIQMTQS

SEQ ID NO: 23 - DNA, 23 bp, Homo sapiens
  primer
gatgttgtgatgactcagtctcc

SEQ ID NO: 24 - protein, 7 aa, Homo sapiens
DVVMTQS

SEQ ID NO: 25 - DNA, 23 bp, Homo sapiens
  primer
gaaattgtgttgacgcagtctcc

SEQ ID NO: 26 - protein, 7 aa, Homo sapiens
EIVLTQS

SEQ ID NO: 27 - DNA, 23 bp, Homo sapiens
  primer
gacatcgtgatgacccagtctcc

SEQ ID NO: 28 - protein, 7 aa, Homo sapiens
DIVMTQS

SEQ ID NO: 29 - DNA, 23 bp, Homo sapiens
  primer
gaaacgacactcacgcagtctcc

SEQ ID NO: 30 - protein, 7 aa, Homo sapiens
ETTLTQS

SEQ ID NO: 31 - DNA, 23 bp, Homo sapiens
  primer
gaaattgtgctgactcagtctcc

SEQ ID NO: 32 - protein, 7 aa, Homo sapiens
EIVLTQS

SEQ ID NO: 33 - DNA, 24 bp, Homo sapiens
  primer
acgtttgatttccaccttggtccc

SEQ ID NO: 34 - protein, 8 aa, Homo sapiens
GTKVEIKR

SEQ ID NO: 35 - DNA, 24 bp, Homo sapiens
  primer
acgtttgatctccagcttggtccc

SEQ ID NO: 36 - protein, 8 aa, Homo sapiens
GTKLEIKR

SEQ ID NO: 37 - DNA, 24 bp, Homo sapiens
  primer
acgtttgatatccactttggtccc

SEQ ID NO: 38 - protein, 8 aa, Homo sapiens
GTKVDIKR

SEQ ID NO: 39 - DNA, 24 bp, Homo sapiens
  primer
acgtttgatctccaccttggtccc

SEQ ID NO: 40 - protein, 8 aa, Homo sapiens
GTKVEIKR

SEQ ID NO: 41 - DNA, 24 bp, Homo sapiens
  primer
acgtttaatctccagtcgtgtccc

SEQ ID NO: 42 - protein, 8 aa, Homo sapiens
GTRLEIKR

SEQ ID NO: 43 - DNA, 23 bp, Homo sapiens
  primer
cagtctgtgttgacgcagccgcc

SEQ ID NO: 44 - protein, 7 aa, Homo sapiens
QSVLTQP

SEQ ID NO: 45 - DNA, 23 bp, Homo sapiens
  primer
cagtctgccctgactcagcctgc

SEQ ID NO: 46 - protein, 7 aa, Homo sapiens
QSALTQP

SEQ ID NO: 47 - DNA, 23 bp, Homo sapiens
  primer
tcctatgtgctgactcagccacc

SEQ ID NO: 48 - protein, 7 aa, Homo sapiens
SYVLTQP

SEQ ID NO: 49 - DNA, 23 bp, Homo sapiens
  primer
tcttctgagctgactcaggaccc

SEQ ID NO: 50 - protein, 7 aa, Homo sapiens
SSELTQD

SEQ ID NO: 51 - DNA, 23 bp, Homo sapiens
  primer
cacgttatactgactcaaccgcc

SEQ ID NO: 52 - protein, 7 aa, Homo sapiens
HVILTQP

SEQ ID NO: 53 - DNA, 23 bp, Homo sapiens
  primer
caggctgtgctcactcagccgtc

SEQ ID NO: 54 - protein, 7 aa, Homo sapiens
QAVLTQP

SEQ ID NO: 55 - DNA, 23 bp, Homo sapiens
  primer
aattttatgctgactcagcccca

SEQ ID NO: 56 - protein, 7 aa, Homo sapiens
NFMLTQP

SEQ ID NO: 57 - DNA, 24 bp, Homo sapiens
  Homo sapiens
acctaggacggtgaccttggtccc

SEQ ID NO: 58 - protein, 8 aa, Homo sapiens
GTKVTVLG

SEQ ID NO: 59 - DNA, 24 bp, Homo sapiens
  primer
acctaggacggtcagcttggtccc

SEQ ID NO: 60 - protein, 8 aa, Homo sapiens
GTKLTVLG

SEQ ID NO: 61 - DNA, 24 bp, Homo sapiens
  primer
acctaaaacggtgagctgggtccc

SEQ ID NO: 62 - protein, 8 aa, Homo sapiens
GTQLTVLG

SEQ ID NO: 63 - DNA, 375 bp, Homo sapiens
  CDS (1)..(375)
gaggtgcagctggtggagtctgggggaggcctggtcaagcctggggggtccctgagactctcctgtgttgcctctggattcacattcagagtctataccataaactgggtccgtcaggctccagggaaggggctggagtgggtctcatccattagtggaagtggcgctgtgatatattacgctgactcagtgaagggccgcttcaccatctccagagacaactccaagaactcagtgtctctgcaaatgaacagcctgagagccgaagacacggctgtgtatttttgtgcgcgggatggagagatggccacaactccccggaacgactacgctttggacgtctggggccaagggaccacggtcaccgtctcctca

SEQ ID NO: 64 - protein, 125 aa, Homo sapiens
EVQLVESGGGLVKPGGSLRLSCVASGFTFRVYTINWVRQAPGKGLEWVSSISGSGAVIYYADSVKGRFTISRDNSKNSVSLQMNSLRAEDTAVYFCARDGEMATTPRNDYALDVWGQGTTVTVSS

SEQ ID NO: 65 - DNA, 375 bp, Homo sapiens
  CDS (1)..(375)
gaggtgcagctggtggagtctgggggaggcctggtcaagcctggggggtccctgagactctcctgtgttgcctctggattcacattcagagtctataccataaactgggtccgtcaggctccagggaaggggctggagtgggtctcatccactagtggaagtggcgctgtgatatattacgctgactcagtgaagggccgcttcaccatctccagagacaactccaagaactcagtgtctctgcaaatgaacagcctgagagccgaagacacggctgtgtatttttgtgcgcgggatggagagatggccacaactccccggaacgactacgctttggacgtctggggccaagggaccacggtcaccgtctcctca

SEQ ID NO: 66 - protein, 125 aa, Homo sapiens
EVQLVESGGGLVKPGGSLRLSCVASGFTFRVYTINWVRQAPGKGLEWVSSTSGSGAVIYYADSVKGRFTISRDNSKNSVSLQMNSLRAEDTAVYFCARDGEMATTPRNDYALDVWGQGTTVTVSS

SEQ ID NO: 67 - DNA, 375 bp, Homo sapiens
  CDS (1)..(375)
gaggtgcagctggtgcagtctgggggaggcctggtcaagcctggggggtccctgagactctcctgtgttgcctctggattcacattcagagtctataccataaactgggtccgtcaggctccagggaaggggctggagtgggtctcatccattagtggaagtggcgctgtgatatattacgctgactcagtgaatggccgcttcaccatctccagagacaactccaagaactcagtgtctctgcaaatgaacagcctgagagccgaagacacggctgtgtatttttgtgcgcgggatggagagatggccacaactccccggaacgactacgctttggacgtctggggccaagggaccacggtcaccgtctcctca

SEQ ID NO: 68 - protein, 125 aa, Homo sapiens
EVQLVQSGGGLVKPGGSLRLSCVASGFTFRVYTINWVRQAPGKGLEWVSSISGSGAVIYYADSVNGRFTISRDNSKNSVSLQMNSLRAEDTAVYFCARDGEMATTPRNDYALDVWGQGTTVTVSS

SEQ ID NO: 69 - DNA, 336 bp, Homo sapiens
  CDS (1)..(336)
cagtctgtgttgacgcagccgccctcagtgtctggggccccagggcagagggtcaccatctcctgcactgggagcagctccaacatcggggcaggttttgatgttcactggtaccagcagcttccaggaacagcccccaaactcctcatctatgttaataccaatcggccctcaggggtccctgaccggttctctggctccaagtctggcacctcagcctccctggccatcactgggctccaggctgaggatgaggctgattattactgccagtcctatgacagcagcctgagtggttgggtattcggcggggggacccagctcaccgtcctaggt

SEQ ID NO: 70 - protein, 112 aa, Homo sapiens
QSVLTQPPSVSGAPGQRVTISCTGSSSNIGAGFDVHWYQQLPGTAPKLLIYVNTNRPSGVPDRFSGSKSGTSASLAITGLQAEDEADYYCQSYDSSLSGWVFGGGTQLTVLG

SEQ ID NO: 71 - DNA, 375 bp, Homo sapiens
  CDS (1)..(375)
  misc_feature (16)..(16): s=g or c
gaggtgcagctggtgsagtctgggggaggcctggtcaagcctggggggtccctgagactctcctgtgttgcctctggattcacattcagagtctataccataaactgggtccgtcaggctccagggaaggggctggagtgggtctcatccaytagtggaagtggcgctgtgatatattacgctgactcagtgaakggccgcttcaccatctccagagacaactccaagaactcagtgtctctgcaaatgaacagcctgagagccgaagacacggctgtgtatttttgtgcgcgggatggagagatggccacaactccccggaacgactacgctttggacgtctggggccaagggaccacggtcaccgtctcctca

SEQ ID NO: 72 - protein, 125 aa, Homo sapiens
  misc_feature (6)..(6): The ′Xaa′ at location 6 stands for Glu, or Gln.
EVQLVXSGGGLVKPGGSLRLSCVASGFTFRVYTINWVRQAPGKGLEWVSSXSGSGAVIYYADSVXGRFTISRDNSKNSVSLQMNSLRAEDTAVYFCARDGEMATTPRNDYALDVWGQGTTVTVSS

SEQ ID NO: 73 - DNA, 15 bp, Homo sapiens
  CDS (1)..(15)
gtctataccataaac

SEQ ID NO: 74 - protein, 5 aa, Homo sapiens
VYTIN

SEQ ID NO: 75 - DNA, 51 bp, Homo sapiens
  CDS (1)..(51)
  misc_feature (5)..(5): y=t or c
tccaytagtggaagtggcgctgtgatatattacgctgactcagtgaakggc

SEQ ID NO: 76 - protein, 17 aa, Homo sapiens
  misc_feature (2)..(2): The ′Xaa′ at location 2 stands for Thr, or Ile.
SXSGSGAVIYYADSVXG

SEQ ID NO: 77 - DNA, 48 bp, Homo sapiens
  CDS (1)..(48)
gatggagagatggccacaactccccggaacgactacgctttggacgtc

SEQ ID NO: 78 - protein, 16 aa, Homo sapiens
DGEMATTPRNDYALDV

SEQ ID NO: 79 - DNA, 42 bp, Homo sapiens
  CDS (1)..(42)
actgggagcagctccaacatcggggcaggttttgatgttcac

SEQ ID NO: 80 - protein, 14 aa, Homo sapiens
TGSSSNIGAGFDVH

SEQ ID NO: 81 - DNA, 21 bp, Homo sapiens
  CDS (1)..(21)
gttaataccaatcggccctca

SEQ ID NO: 82 - protein, 7 aa, Homo sapiens
VNTNRPS

SEQ ID NO: 83 - DNA, 33 bp, Homo sapiens
  CDS (1)..(33)
cagtcctatgacagcagcctgagtggttgggta

SEQ ID NO: 84 - protein, 11 aa, Homo sapiens
QSYDSSLSGWV

SEQ ID NO: 85 - DNA, 328 bp, Homo sapiens
  CDS (2)..(328)
gggaggcctggtcaagcctggggggtccctgagactctcctgtgttgcctctggattcacattcagagtctataccataaactgggtccgtcaggctccagggaaggggctggagtgggtctcatccattagtggaagtggcgctgtgatatattacgctgactcagtgaagggccgcttcaccatctccagagacaactccaagaactcagtgtctctgcaaatgaacagcctgagagccgaagacacggctgtgtatttttgtgcgcgggatggagagatggccacaactccccggaacgactacgctttggacgtctggggccaa

SEQ ID NO: 86 - protein, 109 aa, Homo sapiens
GGLVKPGGSLRLSCVASGFTFRVYTINWVRQAPGKGLEWVSSISGSGAVIYYADSVKGRFTISRDNSKNSVSLQMNSLRAEDTAVYFCARDGEMATTPRNDYALDVWGQ

SEQ ID NO: 87 - DNA, 289 bp, Homo sapiens
  CDS (2)..(289)
ctcagtgtctggggccccagggcagagggtcaccatctcctgcactgggagcagctccaacatcggggcaggttttgatgttcactggtaccagcagcttccaggaacagcccccaaactcctcatctatgttaataccaatcggccctcaggggtccctgaccggttctctggctccaagtctggcacctcagcctccctggccatcactgggctccaggctgaggatgaggctgattattactgccagtcctatgacagcagcctgagtggttgggtattcggcggg

SEQ ID NO: 88 - protein, 96 aa, Homo sapiens
SVSGAPGQRVTISCTGSSSNIGAGFDVHWYQQLPGTAPKLLIYVNTNRPSGVPDRFSGSKSGTSASLAITGLQAEDEADYYCQSYDSSLSGWVFGG

SEQ ID NO: 89 - DNA, 33 bp, Artificial sequence
  primer
ccccttaagcgaggtgcagctggtggagtctgg

SEQ ID NO: 90 - DNA, 33 bp, Artificial sequence
  primer
cccgctagctgaggagacggtgaccgtggtccc

SEQ ID NO: 91 - DNA, 47 bp, Artificial sequence
  primer
cccccctgtacaggctctgaggcccagtctgtgttgacgcagccgcc

SEQ ID NO: 92 - DNA, 47 bp, Artificial sequence
  primer
cccccctgtacaggctctgaggcccagtctgccctgactcagcctgc

SEQ ID NO: 93 - DNA, 47 bp, Artificial sequence
  primer
cccccctgtacaggctctgaggcctcctatgtgctgactcagccacc

SEQ ID NO: 94 - DNA, 47 bp, Artificial sequence
  primer
cccccctgtacaggctctgaggcctcttctgagctgactcaggaccc

SEQ ID NO: 95 - DNA, 47 bp, Artificial sequence
  primer
cccccctgtacaggctctgaggcccacgttatactgactcaaccgcc

SEQ ID NO: 96 - DNA, 47 bp, Artificial sequence
  primer
cccccctgtacaggctctgaggcccaggctgtgctcactcagccgtc

SEQ ID NO: 97 - DNA, 47 bp, Artificial sequence
  primer
cccccctgtacaggctctgaggccaattttatgctgactcagcccca

SEQ ID NO: 98 - DNA, 29 bp, Artificial sequence
  primer
ggggggcctaggacggtgaccttggtccc

SEQ ID NO: 99 - DNA, 29 bp, Artificial sequence
  primer
gggggacctaggacggtcagcttggtccc

SEQ ID NO: 100 - DNA, 29 bp, Artificial sequence
  primer
ggggggcctaggacggtgagctgggtccc

SEQ ID NO: 101 - DNA, 22 bp, Artificial sequence
  primer
cccactgcttactggcttatcg

SEQ ID NO: 102 - DNA, 18 bp, Artificial sequence
  primer
ggtgctcttggaggaggg

SEQ ID NO: 103 - DNA, 1425 bp, Homo sapiens
  CDS (1)..(1422)
  misc_feature (73)..(73): s=g or c
atgaaacacctgtggttcttcctcctgctggtggcagctcccagatgggtcttaagcgaggtgcagctggtgsagtctgggggaggcctggtcaagcctggggggtccctgagactctcctgtgttgcctctggattcacattcagagtctataccataaactgggtccgtcaggctccagggaaggggctggagtgggtctcatccaytagtggaagtggcgctgtgatatattacgctgactcagtgaakggccgcttcaccatctccagagacaactccaagaactcagtgtctctgcaaatgaacagcctgagagccgaagacacggctgtgtatttttgtgcgcgggatggagagatggccacaactccccggaacgactacgctttggacgtctggggccaagggaccacggtcaccgtctcctcagctagtaccaagggcccatccgtcttccccctggcaccctcctccaagagcacctctgggggcacagcggccctgggctgcctggtcaaggactacttccctgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 104 - protein, 474 aa, Homo sapiens
  misc_feature (25)..(25): The ′Xaa′ at location 25 stands for Glu, or Gln.
MKHLWFFLLLVAAPRWVLSEVQLVXSGGGLVKPGGSLRLSCVASGFTFRVYTINWVRQAPGKGLEWVSSXSGSGAVIYYADSVXGRFTISRDNSKNSVSLQMNSLRAEDTAVYFCARDGEMATTPRNDYALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 711 bp, Homo sapiens
  CDS (1)..(708)
atggcctggattcctctacttctccccctcctcactctctgtacaggctctgaggcccagtctgtgttgacgcagccgccctcagtgtctggggccccagggcagagggtcaccatctcctgcactgggagcagctccaacatcggggcaggttttgatgttcactggtaccagcagcttccaggaacagcccccaaactcctcatctatgttaataccaatcggccctcaggggtccctgaccggttctctggctccaagtctggcacctcagcctccctggccatcactgggctccaggctgaggatgaggctgattattactgccagtcctatgacagcagcctgagtggttgggtattcggcggggggacccagctcaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 106 - protein, 236 aa, Homo sapiens
MAWIPLLLPLLTLCTGSEAQSVLTQPPSVSGAPGQRVTISCTGSSSNIGAGFDVHWYQQLPGTAPKLLIYVNTNRPSGVPDRFSGSKSGTSASLAITGLQAEDEADYYCQSYDSSLSGWVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2009/0053300 A1
(43) Pub. Date: Feb. 26, 2009

(54) MONOCLONAL ANTIBODY, GENE ENCODING THE ANTIBODY, HYBRIDOMA, PHARMACEUTICAL COMPOSITION, AND DIAGNOSTIC REAGENT

(75) Inventors: Saiko Hosokawa, Tokyo (JP); Yoko Hirakawa, Tokyo (JP); Kazumasa Fukuda, Tokyo (JP); Rieko Nakamura, Tokyo (JP); Yoshiro Saikawa, Tokyo (JP); Masahiko Aoki, Tokyo (JP); Koichiro Kumai, Tokyo (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: MITSUBISHI TANABE PHARMA CORPORATION, Osaka-shi (JP); KEIO UNIVERSITY, Tokyo (JP)

(21) Appl. No.: 12/279,160

(22) PCT Filed: Feb. 23, 2007
(86) PCT No.: PCT/JP2007/053391

§ 371 Date: Aug. 12, 2008

(30) Foreign Application Priority Data
Feb. 23, 2006 (JP) 2006-046974

(51) Int. Cl. A61K 39/395; C07K 16/00; C12N 15/13; C12N 15/85; A61K 39/44; A61K 9/127; C12N 5/10; C12N 5/16

(52) U.S. Cl. 424/450; 530/388.15; 536/23.53; 435/320.1; 435/325; 435/326; 424/142.1

(57) ABSTRACT

Disclosed is a monoclonal antibody which has a heavy chain variable region containing amino acid sequences depicted in SEQ ID NOs:74, 76 and 78 and a light chain variable region containing amino acid sequences depicted in SEQ ID NOs:80, 82 and 84. The monoclonal antibody can be used as a cancer therapeutic agent which acts selectively on a cancer tissue of non-small lung cancer, pancreatic cancer, gastric cancer or the like.

### TECHNICAL FIELD

The present invention relates to a novel monoclonal antibody useful for diagnosis and therapy of cancer, and a DNA encoding such a monoclonal antibody, a hybridoma producing such an antibody, and a pharmaceutical composition and a diagnostic reagent, each of which contains such an antibody.

### BACKGROUND ART

In the field of cancer therapy, targeting therapy against a specific type of cancer cell has been studied so far for the treatment of solid cancer on which no therapeutic agent shows sufficient effect. In such targeting therapy, a monoclonal antibody that specifically recognizes cancer cells is effective. However, the use of a mouse monoclonal antibody has some problems such as difficulty of repetitive administration because of side effects such as anaphylaxis caused by an immune response (Non-patent document 1).

For solving such problems, attempts have been conducted to obtain monoclonal antibodies with reduced side effects. A technology for producing a chimeric antibody in which constant region is replaced with that of human antibody by genetic engineering, a technology for producing a humanized antibody in which all regions except for hypervariable regions are replaced with those of human antibody, etc. are known. However, a complete human monoclonal antibody has been desired from the viewpoint of reducing side effects. As a method of obtaining a complete human monoclonal antibody, there is a hybridoma method using a lymphocyte derived from human (Non-patent document 2). Although there is a few report about a human monoclonal antibody that reacts cancer cells (Patent document 1), preparation of human monoclonal antibodies which adequately react with cancer cells has been still very difficult because of the reasons that it is very difficult to conduct passive immunity for the purpose of obtaining human B cells which produce a desired antibody, and that any efficient methodology which allows infinite reproduction of antibody-producing cells has not been established yet.

Even under such circumstances, some monoclonal antibodies which exhibit a killing-effect or anti-proliferative effect on specific cancer cells by itself or in combination with anti-cancer drugs have been developed using a humanized antibody technology or the like. In recent years, application of an anti-Her2-humanized antibody (HERCEPTIN) to breast cancer (Non-patent document 3), clinical trials using an anti-EGF receptor antibody (Non-patent document 4), or an anti-VEGF (vascular endothelial growth factor) antibody (Non-patent document 5), and the like have been reported. However, any antibody, which can be used for targeting therapy for cancers including a non-small cell lung cancer from which many patients are suffering or refractory cancers such as pancreatic cancer, has not yet been developed. For treating such types of cancer, the acquisition of a monoclonal antibody having high specificity to cancer tissue with reduced side effects has been desired.

Patent document 2 discloses a monoclonal antibody having high specificity to cancer tissues as such an antibody, more antibodies has been desired to be obtained.

[Patent document 1] JP3236667
[Patent document 2] JP2005-040126
[Non-patent document I] Proc. Natl. Acad. Sci. U.S.A. vol. 86, p4220, 1989
[Non-patent document 2] Cancer Res. vol. 45, p263, 1985
[Non-patent document 3] Oncology vol. 63 Suppl 1, pp 25-32, 2002
[Non-patent document 4] Semin Oncol. vol. 29, No. 5 Suppl 14, pp 18-30, 2002
[Non-patent document 5] Semin Oncol. vol. 29, No. 6 Suppl 16, pp 10-14, 2002
[Non-patent document 6] Mol Cell Biol. 1987, vol. 7, No. 11, p3908-15

### DISCLOSURE OF THE INVENTION

An object of the present invention is to provide a monoclonal antibody useful for diagnosis and treatment of cancer, particularly of non-small cell lung cancer, pancreatic cancer, and gastric cancer with reduced side effects.

The inventors of the present invention have made extensive studies to provide a monoclonal antibody that can be used in targeting therapy on cancer tissues. As a result, they prepared hybridoma cells that produce a novel human monoclonal antibody capable of specifically recognizing cancer cells such as HLC-1, PANC-1, HT29 and MKN45, and found that anti-cancer drug useful in targeting therapy can be obtained by using such an antibody. Accordingly, they completed the present invention.

That is, the present invention provides the followings.

(1) A monoclonal antibody, wherein variable region of heavy chain of said monoclonal antibody comprises amino acid sequences of SEQ ID NOS: 74, 76 and 78.

(2) The monoclonal antibody according to (1), wherein the variable region of the heavy chain comprises an amino acid sequence of SEQ ID NO: 72.

(3) The monoclonal antibody according to (1), wherein the variable region of the heavy chain comprises an amino acid sequence of SEQ ID NO: 104.

(4) A monoclonal antibody, wherein variable region of the light chain of said monoclonal antibody comprises amino acid sequences of SEQ ID NOS: 80, 82 and 84.

(5) The monoclonal antibody according to (4), wherein the variable region of the light chain comprises an amino acid sequence of SEQ ID NO: 70.

(6) The monoclonal antibody according to (4), wherein the variable region of the light chain comprises an amino acid sequence of SEQ ID NO: 106.

(7) A monoclonal antibody, wherein variable region of the heavy chain of said monoclonal antibody comprises amino acid sequences of SEQ ID NOS: 74, 76 and 78, and variable region of the light chain of said monoclonal antibody comprises amino acid sequences of SEQ ID NOS: 80, 82 and 84.

(8) The monoclonal antibody according to (7), wherein the variable region of the heavy chain contains an amino acid sequence of SEQ ID NO: 72, and the variable region of the light chain comprises an amino acid sequence of SEQ ID NO: 70.

(9) The monoclonal antibody according to (7), wherein the variable region of the heavy chain comprises an amino acid sequence of SEQ ID NO: 104, and the variable region of the light chain comprises an amino acid sequence of SEQ ID NO: 106.

(10) The monoclonal antibody according to any one of (1) to (9), wherein said monoclonal antibody is a human antibody.

(11) A DNA which encodes the monoclonal antibody according to any one of (1) to (10).

(12) The DNA according to (11), wherein a region that encodes variable region of the heavy chain comprises nucleotide sequences of SEQ ID NOS: 73, 75 and 77, and a region that encodes variable region of the light chain comprises nucleotide sequences of SEQ ID NOS: 79, 81 and 83.

(13) The DNA according to (11) or (12), wherein the region that encodes the variable region of the heavy chain comprises a nucleotide sequence of SEQ ID NO: 71, and the region that encodes the variable region of the light chain comprises a nucleotide sequence of SEQ ID NO: 69.

(14) The DNA according to (11) or (12), wherein a region that encodes the variable region of the heavy chain comprises a nucleotide sequence of SEQ ID NO: 103, and a region that encodes the variable region of the light chain comprises a nucleotide sequence of SEQ ID NO: 105.

(15) A recombinant vector, comprising the DNA according to any one of (11) to (14).

(16) A transformant, comprising the recombinant vector according to (15).

(17) A hybridoma which produces the monoclonal antibody according to any one of (1) to (10).

(18) A pharmaceutical composition, comprising the monoclonal antibody according to any one of (1) to (10).

(19) The pharmaceutical composition according to (18), which is a composition for cancer treatment.

(20) The pharmaceutical composition according to (19), wherein the composition for cancer treatment is a composition for treatment of one or two or more cancers selected from the group consisting of non-small cell lung cancer, pancreatic cancer and gastric cancer.

(21) The pharmaceutical composition according to any one of (18) to (20), wherein the monoclonal antibody is anchored on a surface of a liposome in which a toxin or an anti-cancer drug is encapsulated.

(22) A diagnostic reagent, which comprises the monoclonal antibody according to any one of (1) to (10).

### BRIEF DESCRIPTION OF THE DRAWINGS

FIG. 1 is a diagram (photograph) showing the reactivity of the 020630-18-1 antibody to various tissue slices.

FIG. 2 is a diagram (photograph) showing the binding activity of the 1F8 antibody to the surface of living lung cancer cell and the surface of living normal lung cell, respectively.

FIG. 3 is a diagram showing anti-proliferative effects of the 1F8 antibody on the cultured cancer cell lines HLC-1, PANC-1, and MKN45, respectively.

FIG. 4 is a diagram (photograph) showing the binding activity of γ-1F8 antibody to the surface of fixed cancer cell.

### DETAILED DESCRIPTION OF THE PREFERRED EMBODIMENTS

Hereinafter, the present invention will be described in detail.

### <1> Monoclonal Antibody of the Present Invention

The monoclonal antibody of the present invention is a monoclonal antibody that specifically recognizes a cancer cell-specific antigen.

Specific examples of the monoclonal antibody of the present invention include antibodies in which variable region of the heavy chain comprises amino acid sequences of SEQ ID NOS: 74, 76 and 78 and variable region of the light chain comprises amino acid sequences of SEQ ID NOS: 80, 82 and 84. In the amino acid sequence of SEQ ID NO: 76, the amino acid at position 2 may be Ile or Thr, and the amino acid at position 16 may be Lys or Asn. That is, there are four kinds of sequences of (2Ile, 16Lys), (2Ile, 16Asn), (2Thr, 16Lys) and (2Thr, 16Asn) as a variable region of the heavy chain.

The above six kinds of sequences are the sequences of the regions called as “hypervariable regions” in variable regions of the heavy and light chain. An antibody consists of heavy chains and light chains, and each of these chains is composed of a constant region and a variable region. A variable region comprises hypervariable regions which determine the specificity of immunoglobulin as an antibody and the binding affinity of the antibody to an epitope. Therefore, regions other than hypervariable regions may be derived from any of other antibodies, so far as hypervariable regions of the present invention comprise each of the above sequences. Here, the term “other antibodies” includes antibodies derived from organisms other than human, but antibodies of human origin are preferable in terms of reducing side effects.

In the present invention, a particularly preferable monoclonal antibody may be one comprising an amino acid sequence of SEQ ID NO: 72 in variable region of the heavy chain and an amino acid sequence of SEQ ID NO: 70 in variable region of the light chain. Here, in the sequence of SEQ ID NO: 72, the amino acid at position 51 may be Ile or Thr, and the amino acid at position 65 may be Lys or Asn.

The antibody of the present invention may have substitution, deletion, or addition of one or several amino acids in the sequence of SEQ ID NO: 72 (heavy chain) and 70 (light chain) as long as the antibody has the specificity that it specifically recognizes a cancer cell-specific antigen. Here, the term “several” means preferably two to five, more preferably two to three, particularly preferably two. Such a substitution, deletion, or addition may be introduced into hypervariable regions, but preferably introduced into regions other than hypervariable regions in variable regions.

In the present invention, the term “monoclonal antibody” refers to any of those including monoclonal antibodies and fragments thereof, F(ab′)₂ antibodies, F(ab′) antibodies, short-chain antibodies (scFv), diabodies, and minibodies. When the monoclonal antibody contains a constant region, amino acid sequences of its constant regions of the heavy and light chains are preferably one of those described in Nucleic Acids Research vol. 14, p1779, 1986; The Journal of Biological Chemistry vol. 257, p1516, 1982; and Cell vol. 22, p197, 1980. The antibody of the present invention comprising constant and variable regions may be, for example, one comprising an amino acid sequence of SEQ ID NO: 104 (heavy chain) and SEQ ID NO: 106 (light chain).

The monoclonal antibody of the present invention can be obtained by culturing a hybridoma producing the antibody of the present invention in a culture medium, for example, a RPMI1640 medium that contains fetal bovine serum. Alternatively, it can be obtained by preparing a gene (e.g., a gene comprising SEQ ID NO: 103 (heavy chain) or 105 (light chain)), in which a DNA encoding a constant region of heavy chain or light chain is ligated to a DNA encoding each variable region (e.g. DNA comprising SEQ ID NOS: 71 or 69), by means of a PCR method or a chemical synthesis; inserting the obtained gene into a conventionally-used expression vector (e.g., pcDNA3.1 (Invitrogen)) capable of expressing the gene; expressing the gene in a host cell such as a CHO cell (Chinese hamster ovary cell) or Escherichia coli to produce the antibody; and purifying the obtained antibody from the culture medium using a Protein A column or the like.

### <2> DNA of the Present Invention

The DNA of the present invention is a DNA that encodes the monoclonal antibody of the present invention. Examples thereof include a DNA that comprises a region encoding variable region of the heavy chain which comprises amino acid sequences of SEQ ID NOS: 74, 76 and 78; and a region encoding variable region of light chain which comprises amino acid sequences of SEQ ID NOS: 80, 82 and 84. Preferably, such a DNA comprises a region encoding variable region of the heavy chain which comprises nucleotide sequences of SEQ ID NOS: 73, 75 and 77; and a region encoding variable region of the light chain which comprises nucleotide sequences of SEQ ID NOS: 79, 81 and 83. Here, in the sequence of SEQ ID NO: 75, the nucleotide at position 5 may be either “t” or “c”, and the nucleotide at position 48 may be either “g” or “t”.

The hypervariable regions encoded by those DNA sequences are regions that define the specificity of the antibody, so that sequences encoding the other regions may be those derived from other antibodies. Here, the term “other antibodies” includes antibodies derived from organisms other than human, but, antibodies of human origin are preferable in terms of reducing side effect.

In the present invention, particularly preferable DNA may be one comprising a sequence encoding an amino acid sequence of SEQ ID NO: 72 in variable region of the heavy chain and a sequence encoding an amino acid sequence of SEQ ID NO: 70 in variable region of the light chain. Of those, a particularly preferable DNA may be one comprising a nucleotide sequence of SEQ ID NO: 71 encoding variable region of the heavy chain and a nucleotide sequence of SEQ ID NO: 69 encoding variable region of the light chain. Here, in the sequence of SEQ ID NO: 71, the nucleotide at position 152 may be either “t” or “c”, and the nucleotide at position 195 may be either “g” or “t”.

Furthermore, the DNA of the present invention may be one which is capable of hybridizing with a DNA comprising nucleotide sequence of SEQ ID NO: 71, and nucleotide sequence of SEQ ID NO: 69 under stringent conditions as long as it encodes a monoclonal antibody that specifically recognizes a cancer cell-specific antigen. Here, stringent conditions include those under which hybridization is performed at a salt concentration corresponding to 60° C., 1×SSC, 0.1% SDS, preferably 0.1×SSC, 0.1% SDS, which corresponds to washing conditions in Southern hybridization.

The DNA of the present invention may be one that encodes all of the constant region and variable region of heavy and light chains. Alternatively, it may be one encoding only the variable regions of the heavy and light chains. When the DNA encodes all of the constant region and variable region, the nucleotide sequences of the constant regions of the heavy and light chains are preferably those described in Nucleic Acids Research vol. 14, p1779, 1986, The Journal of Biological Chemistry vol. 257, p1156, 1982, and Cell vol. 22, p197, 1980. The DNA of the present invention, which encodes the constant region and variable region, encompasses a DNA comprising a nucleotide sequence of SEQ ID NO: 103 (heavy chain) and a nucleotide sequence of SEQ ID NO: 105 (light chain).

The DNA of the present invention can be obtained by the method described below. At first, total RNA is prepared from the cells (e.g., hybridoma cells) of the present invention using a commercially-available RNA extraction kit and then cDNA is synthesized from the total RNA by reverse transcriptase using random primers and the like. Subsequently, using the PCR method in which oligonucleotides each having a sequence conserved in a variable region of heavy chain or light chain of human antibody are used as primers, cDNA encoding such an antibody is amplified. The sequence encoding the constant region can be obtained by amplification of the known sequence by the PCR method. The nucleotide sequence of the DNA can be determined by a conventional method after inserting the DNA into a plasmid for sequence determination. The DNA encoding the monoclonal antibody of the present invention may also be obtained by chemically synthesizing a sequence of variable region or part thereof and linking it to a sequence comprising the constant region.

Furthermore, the present invention provides a recombinant vector comprising the DNA of the present invention, and a transformant containing the recombinant vector. The recombinant vector may be a vector which can be used for gene expression in prokaryotic cells such as Escherichia coli (e.g., pBR322, pUC119 or a derivative thereof), preferable is a vector which can be used for gene expression in eukaryotic cells, and more preferable is a vector which can be used for gene expression in cells derived from a mammal. Examples of the vectors which can be used for gene expression in cells derived from a mammal include a plasmid vector such as pcDNA3.1 (manufactured by Invitrogen Co., Ltd.) and a virus vector such as pDON-AI DNA (manufactured by TAKARA BIO INC.). The transformant to be introduced with the recombinant vector of the present invention may be a prokaryotic cell such as Escherichia coli, but preferable is a eukaryotic cell, and more preferable is a cell derived from a mammal. Examples of the cells derived from a mammal include a Chinese hamster ovary cell (CHO cell).

### <3> Hybridoma of the Present Invention

The hybridoma of the present invention is a hybridoma that produces the monoclonal antibody as described above. Examples of the hybridomas of the present invention include hybridoma strains 020630-18-1 and 020630-18-1-1F8-1, which will be explained in the Examples described later. The hybridoma of the present invention can be obtained by the following method. At first, on the basis of the method of A. Imam et. al (Cancer Research vol. 45, 263, 1985), tumor-infiltrating lymphocytes are isolated from the tumor tissue removed from a patient diagnosed with gastric cancer and then the cells containing the lymphocytes are fused with mouse myeloma cells using polyethylene glycol to obtain hybridoma cells. Subsequently, enzyme immunoassay is carried out using the supernatant of the obtained hybridoma and then the hybridoma cells that produce antibodies that positively react to various cancer cell lines fixed with paraformaldehyde are selected, followed by cloning the obtained hybridomas by limiting dilution.

### <4> Pharmaceutical Composition of the Present Invention

The pharmaceutical composition of the present invention comprises the monoclonal antibody of the present invention together with a pharmaceutically acceptable carrier. Examples of the pharmaceutically-acceptable carriers include soluble carriers such as known buffers which can be physiologically acceptable (e.g., phosphate buffer) or solid-state carriers such as latex beads.

The pharmaceutical composition of the present invention is suitably used as a therapeutic agent for cancer, particularly for non-small cell lung cancer, pancreatic cancer, and gastric cancer. In addition, the pharmaceutical composition of the present invention may be a composition using the cell-killing effect and anti-proliferative effect of the monoclonal antibody itself, or may be a composition for targeting an anti-cancer drug such as adriamycine to a cancer tissue by binding the anti-cancer drug to the monoclonal antibody of the present invention.

In the present invention, a particularly suitable pharmaceutical composition is one in which the antibody of the present invention is anchored on a liposome containing a toxin, an anti-cancer drug or the like. The liposome used for anchoring the antibody may be composed of a lipid bilayer. Alternatively, the liposome used may be composed of a multiple lipid layers or composed of a single lipid layer. Examples of the constituents of the liposome include phosphatidylcholine, cholesterol and phosphatidyl ethanolamine, and further include phosphatidic acid as a substance for imparting the liposome with electric charge. The ratio of those constituents is, for example, 0.3 to 1 mol, preferably 0.4 to 0.6 mol of cholesterol, 0.01 to 0.2 mol, preferably 0.02 to 0.1 mol of phosphatidylethanolamine, and 0 to 0.4 mol, preferably 0 to 0.15 mol of phosphatidic acid per 1 mol of phosphatidylcholine.

A method of producing the liposome may be any of conventional methods. For instance, it can be produced using a method (Biochimica et Biophysica Acta vol. 812, p55, 1985) in which a mixture of the lipids, from which a solvent has been removed, is emulsified by a homogenizer or the like and then subjected to freeze-thawing to obtain a multilamellar liposome, followed by adjustment of pore size of the liposome appropriately by ultrasonication, high-speed homogenization, or pressure filtration through a membrane having uniform-size pores. Preferably, the liposome has a particle size of 30 to 200 nm.

Examples of the pharmaceutical agents to be encapsulated in the liposome include: carcinostatic agents such as adriamycin, daunomycin, mitomycin, cisplatin, vincristine, epirubicin, methotrexate, 5-Fu (5-Fluorouracil) and aclacinomycin; toxins such as ricin A and diphtheria toxin; and antisense RNA. Encapsulation of the agent into liposome may be accomplished by hydration of the lipids with an aqueous solution of the agent. In addition, adriamycin, daunomycin and epirubicin may be encapsulated into the liposome by a remote-loading method using pH gradient (Cancer Res. vol. 49 p5922, 1989).

Examples of methods of anchoring the monoclonal antibody on the surface of the liposome include a method in which a purified antibody is coupled with a hydrophobic substance for anchoring the antibody on the liposome, and a method in which an antibody is cross-linked to phosphatidyl ethanolamine using glutaraldehyde. More preferable method is a method in which a liposome containing a lipid into which a maleimide group has been introduced is prepared and an anti-cancer agent or toxin is encapsulated therein, and allowing it to react with a thiolated antibody to thereby anchor the antibody on the surface of the liposome. In addition, a water-soluble polymer derivative that contains a reactive site for an amino group, and a thiol- or intrinsic thiol-group moiety can be preferably used (JP 11-152234A). On the other hand, the surface of the liposome may be modified by allowing the remaining maleimide group to react with a thiolated polyalkylene glycol moiety.

Examples of the methods for introducing a thiol-group into the antibody include a method employing N-succinimidyl-3-(2-pyridyldithio)propionate (SPDP) or compounds such as iminothiolane and mercaptoalkylimidate, which is usually used for thiolation of protein, for introducing a thiol-group to the amino group of the antibody, or a method in which an intrinsic dithiol group of the antibody is reduced to form a thiol group. The method using an intrinsic thiol group is preferable from the view point of maintaining the activity of the antibody. Furthermore, the antibody may be treated with an enzyme such as pepsin to form F(ab′)₂ and then reduced with dithiothreitol (DTT) and the like to form F(ab′), which provides one to three thiol groups for binding to the liposome. The binding of the thiolated antibody to the maleimide group-containing liposome may be accomplished by reacting them in a neutral buffer at pH 6.5 to 7.5 for 2 to 16 hours.

The pharmaceutical for cancer treatment of the present invention may be formulated by any of conventional methods such as a dehydration method (JP02-502348A), a method in which a stabilizing agent to obtain a liquid formulation is added (JP64-9331A), and a lyophilization method (JP64-9931A). The pharmaceutical for cancer treatment of the present invention may be administered in intravascularly, intraperitoneally and the like, as local administrations. The dosage thereof can be optimized for the respective drugs encapsulated into the liposome. When the agent is adriamycin, the dosage of adriamycin is 50 mg/kg or less, preferably 10 mg/kg or less, and more preferably 5 mg/kg or less.

### <5> Diagnostic Reagent of the Present Invention

Examples of the diagnostic reagents of the present invention include those taking advantage of the specificity of the antibody of the present invention against cancer cells, particularly a cancer diagnostic reagent comprising the antibody of the present invention, a secondary antibody, a detection substrate, and other components.

### EXAMPLES

Hereinafter, the present invention will be described in more detail with reference to examples. However, the present invention is not limited to these examples without departing from the scope of the present invention.

(1) Preparation of Hybridomas by Cell Fusion between Lymphocytes derived from Regional Lymph Node Cancer from a Cancer Patient and Mouse Myeloma

### (1)-1: Preparation of Lymphocytes

In a plate filled with a culture medium B (culture medium A (RPM11640 or e-RDF+50 μg/ml gentamicin sulfate) supplemented with 10% fetal calf serum (FCS)), lymphocytes separated from regional lymph node cancer exenterated from a patient with gastric cancer were dispersed on a metal mesh. The cell suspension was centrifuged at 3,000 rpm for 5 minutes and 4.8×10⁷ lymphocytes derived from regional lymph node cancer was obtained.

### (1)-2: Cell Fusion

The lymphocytes derived from regional lymph node cancer were fused to mouse myeloma cells (the number thereof being approximately the same as the number of the lymphocytes) using polyethylene glycol 1500 (Roche Diagnostics) according to a standard method (Cancer Research vol. 45, 263, 1985). The fused cells were suspended in a culture medium B so that the cell density becomes 5×10⁵ cells/ml. The suspension was dispensed in a 96-well plate at 100 μl/well and cultured at 37° C. in CO₂ incubator. On the second day, the culture medium B supplemented with 10 μM hypoxanthine, 0.04 μM aminopterin, and 1.6 μM thymidine (culture medium B supplemented with HAT) was added to each of the wells at 100 μl/well and culture was continued until colonies of hybridomas appeared. As a result, colonies of hybridomas appeared in 23 wells.

### (2) Evaluation of Reactivity of Human Monoclonal Antibody to Cancer Cell Lines

### (2) —1: Cancer Cell Line and its Maintenance

Using the culture supernatant of the obtained hybridomas, reactivity to the fixed gastric cancer cell line MKN45 (Japanese Journal of Cancer and Chemotherapy, vol. 5, p 89, 1978; provided from IBL Co., Ltd.) was tested to select a hybridoma of interest. The cancer cell line was maintained and grown at 37° C. under 5% CO₂ in a culture medium D that was prepared by supplementing 5% FCS to culture medium C (D-MEM/F12+50 μg/ml gentamicin sulfate).

### (2)-2: Measurement of Reactivity to Cancer Cell Line

The above-mentioned cancer cell line was cultured in a 96-well plate for 3 to 4 days until it formed a monolayer. After the supernatant was removed, the plate was washed once with 10 mM phosphate buffer (pH 7.4, 0.15 M NaCl) (PBS) and cells were fixed with 2% paraformaldehyde at room temperature for 20 minutes. After washing five times with PBS, the plate was blocked with 150 μl/well of PBS solution containing 5% BSA (bovine serum albumin). Then, the plate was washed five times with PBS and 50 μl of the hybridoma culture supernatant was added thereto to react at 37° C. for one and half hours. Next, the plate was washed five times with PBS and then added with 50 μl of a horseradish peroxidase-conjugated goat antibody against a human antibody (CAPPEL, 1,000-fold dilution) to react at 37° C. for 1 hour. Subsequently, the plate was washed with PBS containing 0.05% of Tween 20 (PBS-T) and then added with 50 μl/well of phosphate-citrate buffer containing o-phenylenediamine dihydrochloride (5.2%) and H₂O₂ (0.015%). Reaction was conducted at room temperature until color development was observed, followed by the measurement of absorbance at 490 nm with a microphotometer (Nihon Intermed). Cloning of hybridoma was conducted by limiting dilution of the cells obtained from the wells in which reactivity was detected, whereby a hybridoma cell line 020630-18-1 was obtained. Hereinafter, a monoclonal antibody obtained from this cell line is referred to as 020630-18-1 antibody. Further, the cloning of hybridoma was repeated, whereby a hybridoma cell line 020630-18-1-1F8-1 was obtained. Hereinafter, a monoclonal antibody obtained from this cell line is referred to as 1F8 antibody.

### (3) Purification and Labeling of 020630-18-1 and 1F8 Monoclonal Antibodies

### (3)-1: Culture of Hybridomas 020630-18-1 and 020630-18-1-1F8-1 and Purification of 020630-18-1 and 1F8 Monoclonal Antibodies

First, fetal calf serum was allowed to pass through a protein A-glass bead column (PROSEP-A) (bio PROCESSING) to prepare a serum from which substances capable of binding to the PROSEP-A were removed. The culture medium A supplemented with 4-10% of this serum was used to culture the hybridoma 020630-18-1 or 020630-18-1-1F8-1. Next, the culture medium in which the hybridoma 020630-18-1 or hybridoma 020630-18-1-1F8-1 had been cultured was loaded onto the PROSEP-A to thereby adsorb a PROSEP-A-adsorbed polypeptide, which was then eluted to purify the PROSEP-A-adsorbed polypeptide. This PROSEP-A-adsorbed polypeptide was used as a 020630-18-1 antibody or a 1F8 antibody in the subsequent procedures. It was considered that the use of the above-mentioned serum for culture enabled to provide the purified 020630-18-1 antibody or the purified 1F8 antibody with no contamination of substances capable of binding to the PROSEP-A including antibodies derived from serum. The 020630-18-1 antibody and the 1F8 antibody were confirmed to be pure IgG by sodium dodecyl sulfate-polyacrylamide gel electrophoresis (SDS-PAGE) (data not shown).

### (3)-2: Biotinylation of 020630-18-1 Antibody or 1F8 Antibody

After the 020630-18-1 antibody or the 1F8 antibody purified with the PROSEP-A was biotinylated with a biotinylation reagent (Amersham Pharmacia Biotech) according to the manufacturer's instructions, the labeled antibodies were separated from free biotin by gel filtration method.

### (4) Analysis of Reactivity to Various Tissue Sections

### (4)-1: Preparation of Various Tissue Sections

Various cancer cell lines (lung cancer cell lines: HLC-1 (Cancer vol. 67, No. 4, pp 483-492, 1976; provided from Dr. Suzuki in the Medical Department of the Keio University), A549, PC-3, and PC-9 (all of which from IBL Co., Ltd.); pancreatic cancer cell lines: SUIT2 (provided from Dr. Iguchi in National Hospital Kyushu Cancer Center), HPAC (ATCC No. CRL-2119), PANC-1 (ATCC No. CRL-1469), and PK8 (obtained from the Cell Resource Center for Biomedical Research; the Institute of Development, Aging and Cancer of the Tohoku University); gastric cancer cell lines: MKN45, MKN74, and HSC-3 (all of which from IBL Co., Ltd.); and colon cancer cell lines: HT29 (ATCC No. HTB38), DLD-1 (ATCC No. CCL221), LoVo (ATCC No. CCL229), and CoLo205 (ATCC No. CCL222)) were each grown in the culture medium D at 37° C. under 5% CO₂ and transplanted at approximately 1×10⁶ to 1×10⁷ cells subcutaneously in nude mice (CLEA Japan, Inc.) to form tumors. The tumor tissue sections were each prepared by extracting the formed tumors, fixing the tumors with formalin solution according to a standard method, and embedding the tumors with paraffin.

### (4)-2: Detection of Reactivity to Various Tissue Sections

After the prepared various tissue sections were subjected to deparaffinization and blocking procedures according to a standard method, the resulting tissue sections were reacted with the biotinylated 020630-18-1 antibody described in Example (3)-2. DAKO Catalyzed Signal Amplification (CSA) System (DAKO) was used for detection, and reactivity was detected as reddish brown stain by diaminobenzidine. For the immunostained tissue sections, cell nuclei in the tissues were stained in blue with hematoxylin in order to obtain tissue images.

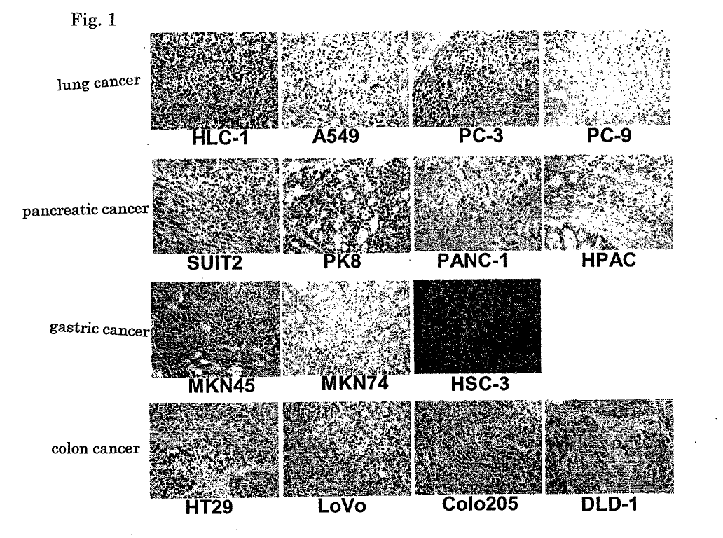

The stained images of various cancer tissue sections of the 020630-18-1 antibody are shown in FIG. 1. It was found that the 020630-18-1 antibody exhibits strong reactivity to the cancer cells in every tumor tissue sections. In particular, nuclei of cancer cells were strongly stained. On the other hand, no reactivity was observed against noncancerous cells in the tissue sections derived from the nude mice.

Further, although not shown in FIG. 1, staining was also conducted in the same procedures as described above by using, as a control, a human antibody purified from human serum with the PROSEP-A column in the same way as the 020630-18-1 antibody. The control antibody showed no specific reactivity to each of the tissue sections.

### (5) Binding Activity of 1F8 Antibody to the Surface of Living Cancer Cells

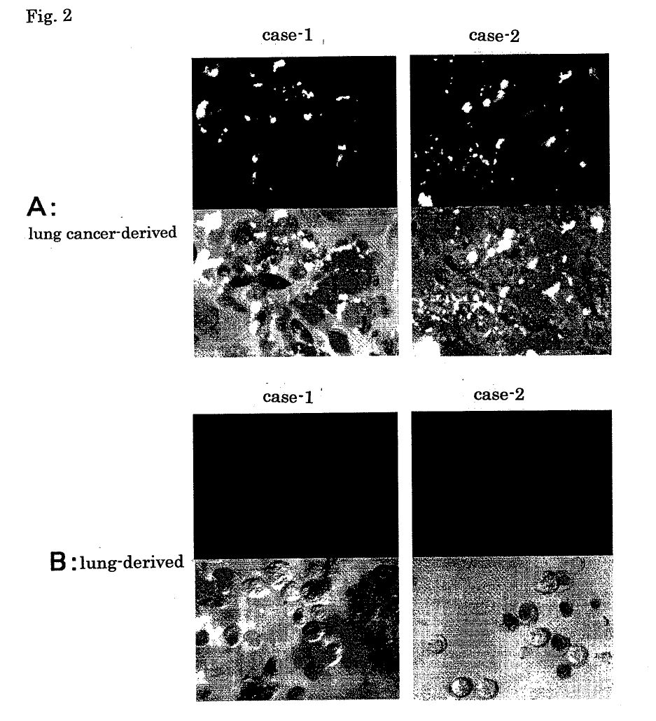

Each nodule of fresh lung cancer tissue and noncancerous lung tissue which was exenterated from a patient with lung cancer by an operation was cut finely with a razor edge, suspended in a culture medium D, passed through a mesh, whereby living cells derived from the tissues were prepared. These living cells were suspended in human serum (containing 0.05% sodium azide), and then the biotinylated 1F8 antibody described in Example (3)-2 was added to the cell solution so that the concentration of the antibody becomes 50 μg/ml, to adjust the total volume of ˜100 μl (number of cells: 10⁵ to 10⁶). The cell suspension was reacted for 60 minutes under ice-cooling condition. This cell suspension was centrifuged (2,000 rpm, 5 minutes) and the supernatant which was a solution containing the antibody was removed. After that, a 20 nM QdotTM565 streptavidin-labeled solution (manufactured by Quantm Dot Corporation) (containing 0.05% sodium azide) was added and reacted at room temperature for 30 minutes. After removal of the solution, PBS (containing 0.05% sodium azide) was added and cells were observed with a confocal fluorescence microscope (CSU10, manufactured by Yokogawa Electric Corporation). The binding activity of the 1F8 antibody to the surface of each living cell is shown in FIG. 2. The 1F8 antibody exhibited binding activity to the surfaces of the living cells derived from the lung cancer tissue (FIG. 2-A), but no binding activity to the living cells derived from the noncancerous lung tissue (FIG. 2-B). The human antibody used as a control exhibited no binding activity to all of the cells (data not shown in FIG. 2).

### (6) Analysis of the Effects on Cancer Cell Lines

(6)-1: Cancer Cell Lines and Vascular Endothelial Cells and their Maintenance

As human cancer cell lines, a lung cancer cell line HLC-1, a gastric cancer cell line MKN45, and a pancreatic cancer cell line PANC-1 were used. These cancer cell lines were maintained and grown at 37° C. under 5% CO₂ in a culture medium D.

### (6)-2: Analysis of the Anti-Proliferative Effect on Cancer Cell Lines

The lung cancer cell line HLC-1, the gastric cancer cell line MKN45, and the pancreatic cancer cell line PANC-1 were each diluted with the culture medium C containing 10% human serum and adjusted so that the number of cells in the well becomes 1.5×10³ cells/100 μl/well and the concentration of 1F8 antibody is serially diluted from 100 μg/ml. These cancer cell lines were cultured at 37° C. under 5% CO₂, the culture supernatant was changed so as to be the same conditions as described above once every other day, three times in total, and on the 6th day, the number of living cancer cells was compared by MTT assay (J. Immunol. Methods vol. 70, p257, 1984).

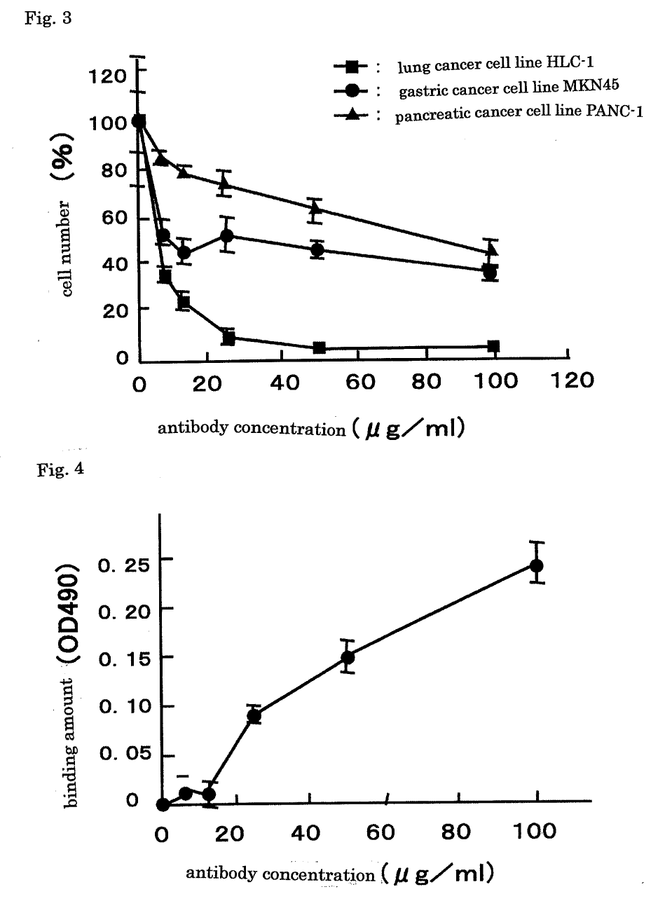

The result is shown in FIG. 3. Anti-proliferative effects were shown by defining the number of cells under control conditions without the addition of the antibody as 100%. The anti-proliferative effects in a concentration-dependent manner were observed in all cancer cell lines by addition of the 1F8 antibody. In particular, the antibody showed a strong anti-proliferative activity to the lung cancer cell line HLC-1. Moreover, no anti-proliferative effect on the cancer cell lines by the addition of the antibody of the same concentration was observed when the human antibody was used as a control (not shown).

### (7) Acquisition of a Gene Encoding Human Monoclonal Antibody 1F8 and Determination of its Nucleotide Sequence

Total RNA was prepared from the 1F8 antibody-producing hybridoma using an RNeasy Protect Mini kit (QIAGEN). Reverse transcription reaction was performed using ThermoScript RT-PCR System (Invitrogen Corporation) and random hexamers as primers to synthesize cDNA.

Based on the known antibody sequence (J Mol. Biol. vol. 222, pp 581-597, 1991), for amplifying variable region of the heavy chain, a mixture of equal amounts of PCR primers (5′ end-primers) including VH1 (SEQ ID NO: 1), VH2 (SEQ ID NO: 3), VH3 (SEQ ID NO: 5), VH4 (SEQ ID NO: 7), VH5 (SEQ ID NO: 9), and VH6 (SEQ ID NO: 11), each corresponding to the N-terminal amino acid sequences (SEQ ID NOS: 2, 4, 6, 8, 10, and 12) conserved in the frame 1 of variable region of the heavy chain of human antibodies and a mixture of equal amounts of PCR primers (3′ end-primers) including JH1 (SEQ ID NO: 13), JH2 (SEQ ID NO: 15), JH3 (SEQ ID NO: 17), and JH4 (SEQ ID NO: 19), each corresponding to the C-terminal amino acid sequences (SEQ ID NOS: 14, 16, 18, and 20) conserved in the frame 4 of variable region of the heavy chain of human antibodies were used as primers for PCR amplification.

A mixture of equal amounts of PCR primers (5′ end-primers) including VK1 (SEQ ID NO: 21), VK2 (SEQ ID NO: 23), VK3 (SEQ ID NO: 25), VK4 (SEQ ID NO: 27), VK5 (SEQ ID NO: 29), and VK6 (SEQ ID NO: 31) each corresponding to the N-terminal amino acid sequences (SEQ ID NOS: 22, 24, 26, 28, 30, and 32) conserved in the frame 1 of variable region of K chain of human antibodies and a mixture of equal amounts of PCR primers (3′ end-primers) including JK1 (SEQ ID NO: 33), JK2 (SEQ ID NO: 35), JK3 (SEQ ID NO: 37), JK4 (SEQ ID NO: 39), and JK5 (SEQ ID NO: 41) each corresponding to the C-terminal amino acid sequences (SEQ ID NOS: 34, 36, 38, 40, and 42) conserved in the frame 4 of variable region of κ chain of human antibodies were used as primers for amplifying variable region of the κ chain.

A mixture of equal amounts of PCR primers (5′ end-primers) including VL1 (SEQ ID NO: 43), VL2 (SEQ ID NO: 45), VL3 (SEQ ID NO: 47), VL4 (SEQ ID NO: 49), VL5 (SEQ ID NO: 51), VL6 (SEQ ID NO: 53), and VL7 (SEQ ID NO: 55) each corresponding to the N-terminal amino acid sequences (SEQ ID NOS: 44, 46, 48, 50, 52, 54, and 56) conserved in the frame 1 of variable region of λ chain of human antibodies and a mixture of equal amounts of PCR primers (3′ end-primers) including JL1 (SEQ ID NO: 57), JL2 (SEQ ID NO: 59), and JL3 (SEQ ID NO: 61) each corresponding to the C-terminal amino acid sequences (SEQ ID NOS: 58, 60, and 62) conserved in the frame 4 of variable region of λ chain of human antibodies were used as primers for amplifying variable region of the λ chain.

PCR was performed using Perkin Elmer Gene Amp PCR System 2400 and ThermoScript RT-PCR System (Invitrogen Corporation) according to the manufacturer's instructions. As a result, the light chain was amplified by PCR with the primers for the λ chain but not amplified with the primers for the K chain. Therefore, it was revealed that the light chain of the antibody produced by the hybridoma was a λ chain.

The amplified DNA fragments each encoding variable region of the heavy or light chain were purified using MiniElute PCR Purification kit (QIAGEN). Next, using TOPO TA cloning kit (Invitrogen Corporation), the purified PCR products were ligated to pCR2.1-TOPO, and the transformation of E. coli was performed. Appeared colonies were picked up and plasmids were purified using QIAGEN Plasmid mini kit (QIAGEN), followed by digestion with EcoRI at 37° C. for 30 minutes and 2% agarose electrophoresis to confirm the insertion of a DNA fragment of interest.

The nucleotide sequences of the inserted DNA fragments were analyzed for several colonies by CEQ 2000 DNA Analysis System (Beckman) using M13 primers. As a result, three sequences including 1F8-H-A (SEQ ID NO: 63), 1F8-H-B (SEQ ID NO: 65), and 1F8-H-C (SEQ ID NO: 67) were identified from the analysis of the heavy chain. A sequence of 1F8-L (SEQ ID NO: 69) was identified from the analysis of the λ chain. By comparing these nucleotide sequences and the corresponding amino acid sequences (heavy chain: SEQ ID NOS: 64, 66, and 68; λ chain: SEQ ID NO: 70), the nucleotide sequences of the variable regions (heavy chain: SEQ ID NO: 71, light chain: SEQ ID NO: 69) and the amino acid sequences (heavy chain: SEQ ID NO: 72, light chain: SEQ ID NO: 70) were determined for each of the heavy and light chains.

A boundary between the hypervariable region (CDR) and the framework was determined with reference to the literature of Kabat et. al. (Sequences of Proteins of Immunological Interest, fifth edition, National Institutes of Health, Bethesda, Md., 1991). As a result, the CDR's of the heavy chain were determined to be HCDR1 (nucleotide sequence: SEQ ID NO: 73, amino acid sequence: SEQ ID NO: 74), HCDR2 (nucleotide sequence: SEQ ID NO: 75, amino acid sequence: SEQ ID NO: 76), and HCDR3 (nucleotide sequence: SEQ ID NO: 77, amino acid sequence: SEQ ID NO: 78). The CDR's of the light chain were determined to be LCDR1 (nucleotide sequence: SEQ ID NO: 79, amino acid sequence: SEQ ID NO: 80), LCDR2 (nucleotide sequence: SEQ ID NO: 81, amino acid sequence: SEQ ID NO: 82), and LCDR3 (nucleotide sequence: SEQ ID NO: 83, amino acid sequence: SEQ ID NO: 84).

### (8) Preparation of γ-1F8. (Recombinant 1F8 Antibody)

(8-1) Preparation of cDNA and Acquisition of 1F8 Variable Region Fragment

The preparation of cDNA from the total RNA (described in Example (7)) and the PCR were carried out using Perkin Elmer Gene Amp PCR System 2400 and ThermoScript RT-PCR System, High Fidelity (Invitrogen) according to the manufacturer's instructions. Random hexamers supplied in the kit were used as primers for preparing the cDNA of the heavy chain.

For amplification of the variable regions of the heavy chain and the λ chain, primers having a restriction enzyme site for ligation to an expression plasmid were used. For amplification of the variable region of the heavy chain, P1 (SEQ ID NO: 89) containing a AflII site at the 5′ terminal and P2 (SEQ ID NO: 90) containing a NheI site at the 3′ terminal were used. For amplification of the variable region of the λ chain, P3 (mixture of equal amounts of SEQ ID NO: 91, 92, 93, 94, 95, 96, and 97) containing a BsrGI site at the 5′ terminal and P4 (mixture of equal amounts of SEQ ID NO: 98, 99, and 100) containing a AvrII site at the 3′ terminal were used.

The DNA fragment of the variable region of each of the amplified heavy chain and the λ chain was purified using QIAquick PCR Purification Kit (QIAGEN). Next, in order to ligate the DNA fragments to the expression vector, the DNA fragments were digested with restriction enzymes. The DNA fragment of the heavy chain was digested with AflII and NheI at 37° C. for 2 hours. The DNA fragment of the λ chain was digested with BsrGI and AvrII at 37° C. for 2 hours. The digested DNA fragments were subjected to 1.5% agarose electrophoresis to purify a DNA fragment of interest using a QIAquick Gel Extraction Kit (QIAGEN).

### (8-2) Cleavage of Expression Plasmid and Acquisition of Plasmid Fragment

For expressing the human IgG1-type recombinant antibody of the 1F8, pEX-G1-sig-dhfr″ containing the sequence of the constant region of the heavy chain and the sequence of the dhfr gene were used as a plasmid for expressing the heavy chain, and pEX-lambda-sig containing the sequence of the constant region of the λ chain was used as a plasmid for expressing the λ chain. The pEX-G1-sig-dhfr″ was digested with AflII and SpeI at 37° C. for 2 hours. The pEX-lambda-sig was digested with BsrGI and AvrII for 2 hours. The digested fragments were subjected to 0.8% agarose electrophoresis to purify a fragment of interest using QIAquick Gel Extraction Kit (QIAGEN).

### (8-3) Ligation of 1F8 Variable Region to the Expression Plasmid

The DNA fragment containing the variable region of the heavy chain of the 1F8 obtained by the above-mentioned method was ligated to the cleaved expression plasmid pEX-G1-sig-dhfr″ using Ligation Kit Ver. 2.1 (TAKARA) according to the manufacturer's instructions, and the obtained plasmid was used to transform E. coli DH5α-T1 (Invitrogen Corporation) according to the manufacturer's instructions. The appeared colonies were picked up and the plasmid was purified using QIAprep Spin Miniprep Kit (QIAGEN). The plasmid containing the nucleotide sequence of the heavy chain was digested with HindIII and NheI at 37° C. for 1.5 hours and subjected to 1% agarose electrophoresis, whereby a desired plasmid pEX-1F8-H was obtained. For the obtained plasmid, the nucleotide sequence was confirmed using P5 (SEQ ID NO: 101) at the 5′ terminal and P6 (SEQ ID NO: 102) at the 3′ terminal. The nucleotide sequence of the structural gene of the heavy chain of the 1F8 recombinant antibody is shown in SEQ ID NO: 103.

On the other hand, the DNA fragment containing the variable region of the λ chain of the 1F8 was ligated to the cleaved expression plasmid pKS-lambda-sig using Ligation Kit Ver. 2.1 (TAKARA) according to the manufacturer's instructions, and the obtained plasmid was used to transform E. coli DH5α-T1 (Invitrogen). The appeared colonies were picked up and the plasmid was purified using QIAprep Spin Miniprep Kit (QIAGEN). The plasmid containing the nucleotide sequence of the λ chain was digested with BsrGI and AvrII at 37° C. for 2 hours and subjected to 1% agarose electrophoresis, whereby a desired plasmid pEX-1F8-L was obtained. For the obtained plasmid, the nucleotide sequence was confirmed using primer P5 at the 5′ terminal. The nucleotide sequence of the structural gene of the λ chain of the 1F8 recombinant antibody is shown in SEQ ID NO: 105.

### (8-3) Ligation of a 1F8 Heavy Chain to a Plasmid Containing 1F8λ Chain

For ligating the heavy chain and λ chain of 1F8 to one plasmid, the plasmids obtained above were digested with restriction enzymes. The pEX-1F8-H was digested with NheI at 37° C. for 1.5 hours and the pEX-1F8-L was digested with NheI and SpeI at 37° C. for 1.5 hours. The resulting fragments were purified using QIAquick PCR Purification Kit (QIAGEN). The fragments were ligated with each other using Ligation Kit Ver. 2.1 (TAKARA) according to the manufacturer's instructions, and the obtained plasmid was used to transform E. coli DH5α-T1 (Invitrogen). The appeared colonies were picked up and the plasmid was purified using QIAprep Spin Miniprep Kit (QIAGEN). The obtained plasmid was digested with NheI at 37° C. for 1 hour and subjected to 1.5% agarose electrophoresis, whereby a plasmid of a desired size was selected. Further, they were digested with HindIII at 37° C. for 1 hour and subjected to 1% agarose electrophoresis to be checked, whereby a desired plasmid pEX-1F8-HL was obtained.

(8-4) Preparation of γ-1F8-Producing Recombinant CHO (DG325) cell lines culturable in a serum-free medium were used as cell lines for the preparation of γ-1F8-producing recombinant. The CHO (DG325) were diluted in CHO-S-SFMII (GIBCO) so as to be 1×10⁶ cells/ml. Thereafter, 2 μg of plasmid pEX-1F8-HL DNA and 6 μl of FuGENE 6 (Roche) were mixed to transfect the CHO (DG325) according to the manufacturer's instructions. At 5.5 hours after transfection, EX-CELL325-PF (Nichirei) was added. After two-day culture, 400 μg/ml of G418 (Promega) and 0 or 25 nM of methotrexate (Sigma) were added to the medium as selection reagents and culture was conducted, followed by cloning, whereby γ-1F8-producing cell line was obtained.
(8-5) Screening of Antibody-Producing Cell Line using ELISA Assay

The screening of the antibody-producing cell was conducted by sandwich ELISA assay. First, a goat anti-human immunoglobulin antibody (CAPPEL) was diluted to 50 μg/ml with PBS and put into a 96-well plate (FALCON) at 50 μl/well to be incubated at 37° C. for 2 hours. After washed five times with PBS, the plate was added with 150 μl/well of PBS solution containing 5% bovine serum albumin (BSA) and blocking was performed. The plate was washed with PBS five times and then added with 50 μl of culture supernatant of the recombinant to react at 37° C. for 2 hours. Next, the plate was washed with PBS five times and then added with 50 μl of horseradish peroxidase (HRP)-conjugated goat antibody against a human antibody (1.000-fold dilution, CAPPEL) to react at 37° C. for 1 hour. Next, the plate was washed with PBS containing 0.05% of Tween 20 (PBS-T) and then added with 50 μl/well of phosphate-citrate buffer containing o-phenylenediamine dihydrochloride (5.2%) and H₂O₂ (0.015%). Reaction was conducted at room temperature until color development was observed, followed by stopping the reaction by adding 50 μl/well of 1N—H₂SO₄. The measurement of absorbance at 490 nm with a microphotometer (Nihon Intermed) was performed, and a cell line having the largest amount of production was selected.

### (8-6) Purification of γ-1F8

The γ-1F8-producing cell line obtained above was cultured in a culture medium A containing 4% serum treated with PROSEP-A and added with G418 (Promega), whereby a culture medium containing the γ-1F8 was obtained. Next, the culture medium was loaded onto the PROSEP-A for adsorbing the γ-1F8 on it. The adsorbed γ-1F8 was then eluted whereby the γ-1F8 was purified. It was confirmed that the γ-1F8 was a pure IgG by SDS-PAGE (not shown).

### (8-7) Confirmation of Binding Activity of γ-1F8 to Cancer Cell Line

The binding activity of the purified γ-1F8 described in the item (8-6) to the HLC-1 cell which was fixed by the same method as described in the item (2-2) was confirmed. The antibodies having respective concentrations were reacted with the fixed cancer cell line at 37° C. for 1 hour. The plate was washed with PBS, added with HRP-conjugated goat antibody against a human antibody (1,000-fold dilution, CAPPEL) to react at 37° C. for 1 hour. The reactivity of γ-1F8 was detected, and as a result, it was confirmed that γ-1F8 exhibited binding activity to HLC-1 cell in a concentration-dependent manner (FIG. 4).

### INDUSTRIAL APPLICABILITY

By using a monoclonal antibody obtained in the present invention, an anti-cancer drug that selectively attacks cancer tissues, particularly non-small cell lung cancer, pancreatic cancer or gastric cancer can be provided. Moreover, when the human monoclonal antibody of the present invention is used, an anti-cancer drug capable of continuous administration with reduced side effects can be provided.

1. A monoclonal antibody, wherein variable region of heavy chain of said monoclonal antibody comprises amino acid sequences of SEQ ID NOS: 74, 76 and 78.

2. The monoclonal antibody according to claim 1, wherein the variable region of the heavy chain comprises an amino acid sequence of SEQ ID NO: 72.

3. The monoclonal antibody according to claim 1, wherein the variable region of the heavy chain comprises an amino acid sequence of SEQ ID NO: 104.

4. A monoclonal antibody, wherein variable region of the light chain of said monoclonal antibody comprises amino acid sequences of SEQ ID NOS: 80, 82 and 84.

5. The monoclonal antibody according to claim 4, wherein the variable region of the light chain comprises an amino acid sequence of SEQ ID NO: 70.

6. The monoclonal antibody according to claim 4, wherein the variable region of the light chain comprises an amino acid sequence of SEQ ID NO: 106.

7. A monoclonal antibody, wherein variable region of the heavy chain of said monoclonal antibody comprises amino acid sequences of SEQ ID NOS: 74, 76 and 78, and variable region of the light chain of said monoclonal antibody comprises amino acid sequences of SEQ ID NOS: 80, 82 and 84.

8. The monoclonal antibody according to claim 7, wherein the variable region of the heavy chain contains an amino acid sequence of SEQ ID NO: 72, and the variable region of the light chain comprises an amino acid sequence of SEQ ID NO: 70.

9. The monoclonal antibody according to claim 7, wherein the variable region of the heavy chain comprises an amino acid sequence of SEQ ID NO: 104, and the variable region of the light chain comprises an amino acid sequence of SEQ ID NO: 106.

10. The monoclonal antibody according to claim 7, wherein said monoclonal antibody is a human antibody.

11. A DNA which encodes the monoclonal antibody according to claim 7.

12. The DNA according to claim 11, wherein a region that encodes variable region of the heavy chain comprises nucleotide sequences of SEQ ID NOS: 73, 75 and 77, and a region that encodes variable region of the light chain comprises nucleotide sequences of SEQ ID NOS: 79, 81 and 83.

13. The DNA according to claim 11, wherein the region that encodes the variable region of the heavy chain comprises a nucleotide sequence of SEQ ID NO: 71, and the region that encodes the variable region of the light chain comprises a nucleotide sequence of SEQ ID NO: 69.

14. The DNA according to claim 11, wherein a region that encodes the variable region of the heavy chain comprises a nucleotide sequence of SEQ ID NO: 103, and a region that encodes the variable region of the light chain comprises a nucleotide sequence of SEQ ID NO: 105.

15. A recombinant vector, comprising the DNA according to claim 11.

16. A transformant, comprising the recombinant vector according to claim 15.

17. A hybridoma which produces the monoclonal antibody according to claim 7.

18. A pharmaceutical composition, comprising the monoclonal antibody according to claim 7.

19. The pharmaceutical composition according to claim 18, which is a composition for cancer treatment.

20. The pharmaceutical composition according to claim 19, wherein the composition for cancer treatment is a composition for treatment of one or two or more cancers selected from the group consisting of non-small cell lung cancer, pancreatic cancer and gastric cancer.

21. The pharmaceutical composition according to claim 18, wherein the monoclonal antibody is anchored on a surface of a liposome in which a toxin or an anti-cancer drug is encapsulated.

22. A diagnostic reagent, which comprises the monoclonal antibody according to claim 7.
